(19) United States
(12) Patent Application Publication
(10) Pub. No.: US 2024/0409572 A1
(43) Pub. Date: Dec. 12, 2024

(54) COMPOUND FOR TREATING NEURODEGENERATIVE DISEASES AND ITS ISOLATION METHOD THEREOF

(71) Applicant: SARAWAK BIODIVERSITY COUNCIL, Kuching, Sarawak (MY)

(72) Inventors: Tiong Chia YEO, Jalan Puncak Borneo Kuching, Sarawak (MY); Julian, Cheng Liang VOONG, Jalan Puncak Borneo Kuching, Sarawak (MY); Noreha Binti MAHIDI, Jalan Puncak Borneo Kuching, Sarawak (MY); Mohd Farith bin KOTA, Jalan Puncak Borneo Kuching, Sarawak (MY); Nuraqilah binti OTHMAN, Jalan Puncak Borneo Kuching, Sarawak (MY); Michele MEJIN, Jalan Puncak Borneo Kuching, Sarawak (MY); Nyuk Fong KON, Jalan Puncak Borneo Kuching, Sarawak (MY); Mohammad Farhan Darin bin AZRI, Jalan Puncak Borneo Kuching, Sarawak (MY); Mitchel Constance ak GEORGE, Jalan Puncak Borneo Kuching, Sarawak (MY)

(21) Appl. No.: 18/259,761

(22) PCT Filed: Dec. 23, 2021
(86) PCT No.: PCT/MY2021/050126

§ 371 Date: Jun. 28, 2023

(30) Foreign Application Priority Data
Dec. 28, 2020 (MY) PI2020007106

(51) Int. Cl. C07H 15/203; A61K 31/7034; A61K 36/062; C12P 17/06; C12R 1/77

(52) U.S. Cl. CPC C07H 15/203; A61K 31/7034; A61K 36/062; C12P 17/06; A61K 2236/19; A61K 2236/33; A61K 2236/35; A61K 2236/55; C12R 2001/77

(57) ABSTRACT

A compound, represented by formula (I):

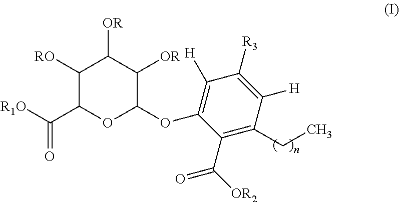

wherein R, R₁, R₂ and R₄ are independently of one another a hydrogen atom or a C₁-C₆ linear or branched alkyl, or a C₁-C₆ linear or branched alkenyl, or a C₁-C₆ linear or branched alkynyl; R₃ is a hydrogen atom, OR₄ or a C₁-C₆ linear or branched alkyl, or a C₁-C₆ linear or branched alkenyl, or a C₁-C₆ linear or branched alkynyl; and n=4 to 11; its functions and isolation process thereof.

### FIELD OF THE INVENTION

The present invention relates to a novel compound for treating neurogenerative diseases and its isolation method thereof.

### BACKGROUND OF THE INVENTION

Neurodegenerative diseases are a group of illnesses that result in the progressive deterioration and/or death of the nervous system which includes both the central nervous system and peripheral nervous system. Examples of neurodegenerative diseases include Parkinson's, Alzheimer's, Huntington's and so on. As most neurodegenerative diseases are irreversible and may lead to problems associated with movement or mental functioning, many efforts have been put into researching and developing new treatment.

To-date, many medications are available in the market for treating neurodegenerative diseases, however, most of these medications may only temporarily reduce the symptoms and patients may be required to be on these medications for life.

For example, some of the current medications for Alzheimer's includes Memantine, an N-methyl D-aspartate (NMDA) antagonist that prevents the excessive buildup of the neurotoxin, glutamate, thus decreasing the symptoms of Alzheimer's, allowing the patient to maintain certain daily functions for a longer period of time. Another medication available is cholinesterase inhibitors such as Donepezil, Rivastigmine and Galantamine, which prevents the breakdown and accumulation of the neurotransmitter acetylcholine in the body, thus alleviating symptoms such as muscular weakness, paralysis, visual memory, processing speed, spatial orientation and so on.

As for Parkinson's disease, one of the common medications available today is Levodopa which is a precursor of dopamine that is used to boost the amount of dopamine in the patient's body, as dopamine is an important neurotransmitter for body movement. Furthermore, another medication which is commonly prescribed together with Levodopa is Carbidopa which functions by allowing higher amounts of Levodopa to cross the blood-brain barrier thus increasing the amount of dopamine to reach the brain, improving cognitive function. Cogentin and Artane are another class of medication that helps in treating Parkinson's disease by restoring the balance between two neurotransmitters, dopamine and acetylcholine, easing symptoms related to movement such as tremors and muscle stiffness.

Huntington's disease, which results in muscle problems can be treated by using medications such as tetrabenazine to help suppress problems associated with voluntary movement, while medications in the form of Risperidone, Haloperidol and Chlorpromazine are taken to help reduce involuntary movement problems.

As there is currently no proven cure for neurodegenerative diseases, it is no surprise that substantive research is continuously conducted to find a curative treatment, whether it is by means of a new treatment pathway or a new medication.

Prolyl Oligopeptidase (POP), also known as Prolyl Endopeptidase (PE) is a ubiquitous post-proline cleaving enzyme that is highly expressed in the brain and facilitates several functions of the central nervous system such as memory, mood and learning (3). POP functions by cleaving short peptides (<30 amino acids) with a high specific cleavage at the carboxyl side of proline. Furthermore, studies have also found that POP can also modulate the functions of protein partners such as neuronal peptides and hormones containing a proline residue (1). As many biologically active compounds contain proline, mainly neuropeptides, there may be a potential approach by inhibiting the activity of POP as curative treatment for neurodegenerative diseases. Moreover, to-date, there are no POP inhibitors as medications available in the market to treat neurodegenerative diseases.

US publication no. 20100099721 A1 and International publication WO 2009007415 A2 discloses hetero-carbonyl compounds, pharmaceutically acceptable salt, polymorph or solvate, including all tautomers and stereoisomers as inhibitor of POP. International publication WO 2014054980 A1 relates to N^{a}-aryl derivatives of aminoacyl-2-cyanopyrrolidine as inhibitors of POP and Dipeptidyl Peptidase-IV for the treatment of several diseases such as type 2 diabetes mellitus and neurogenerative diseases while European publication no. 2730571 A1 discloses compounds derivatives, its preparation methods and pharmaceutical compositions for inhibiting POP enzyme for treating cognitive disorders.

Moreover, a recent study also discloses a similar concept, by evaluating several POP enzyme inhibitors in preclinical trials as potential drugs for the treatment of natural memory deficits that occur with aging or the pathological memory loss characteristic of Alzheimer's disease (2).

From the above documents, it can be observed that the inhibition of POP enzyme may help in the treatment of neurodegenerative diseases. Moreover, these documents have also disclosed potential new compounds as treatments for neurodegenerative diseases by means of inhibiting POP and the compounds mentioned were obtained through synthesis. While synthesis may lead to purer compounds and better yield, there is a possibility that the chemicals used in the synthesis process are hazardous to the human body. Furthermore, some of the chemicals used in the synthesis could also be expensive, which would result in a high overall cost during production. Hence, there is important need to develop synthesis process which are not only safe, but also cost-effective.

Australian patent 2015210849 B2 on the other hand disclose a natural compound, Rosmarinic Acid used to enhance, improve or sustain cognitive health in mammals by inhibiting the POP enzyme. Produced by means of biosynthesis, the compound Rosmarinic Acid was extracted from a plant sample. While biosynthesis may prove to be a more cost-effective way to synthesize new compounds, the isolation of compounds from a plant sample requires the resource and availability of plants, which require time to grow and mature. Furthermore, there is also the risk of the plant bring infected by diseases which may also lead to lower yields.

Thus, there is a need for new compounds as treatment for neurodegenerative diseases which are easily available at a more cost-efficient price.

### SUMMARY OF THE INVENTION

The invention relates to a compound, represented by formula (I):

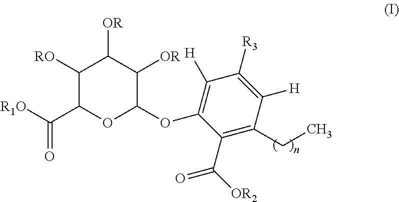

wherein
a) R, R₁, R₂ and R₄ are independently of one another a hydrogen atom or a C₁-C₆ linear or branched alkyl, or a C₁-C₆ linear or branched alkenyl, or a C₁-C₆ linear or branched alkynyl;
b) R₃ is a hydrogen atom, OR₄ or a C₁-C₆ linear or branched alkyl, or a C₁-C₆ linear or branched alkenyl, or a C₁-C₆ linear or branched alkynyl; and,
c) n=4 to 11.

The compound is (2R,3S,4S,5R,6R)-6-(2-carboxy-5-hydroxy-3-undecylphenoxy)-3,4,5-trihydroxytetrahydro-2H-pyran-2-carboxylic acid (FGS03), which is isolated from a fungus, Fusarium sp. strain F274 found in a flower of a Yam plant, Alocasia sp.

The compound is able to inhibit POP enzyme and may be used to treat or improve the quality of living of people suffering from neurodegenerative diseases such as Alzheimer's disease or Parkinson's disease.

A process of isolating the novel compound (2R,3S,4S,5R,6R)-6-(2-carboxy-5-hydroxy-3-undecylphenoxy)-3,4,5-trihydroxytetrahydro-2H-pyran-2-carboxylic acid (FGS03) is also disclosed through the following steps:
a) Fermenting a fungus, Fusarium sp. strain F274 using a fermentation medium;
b) Extracting the compound from the fermentation medium of step (a) using a first organic solvent;
c) Fractioning the compound of step (b) using a first stationary phase and a first mobile phase; and,
d) Purifying the compound of step (c) using a second stationary phase and a second mobile phase.

The detailed embodiments of the invention are further discussed in the sections below.

### BRIEF DESCRIPTION OF THE DRAWINGS

FIG. 1 illustrates the compound of the present invention.

FIG. 2 illustrates the molecular structure of (2R,3S,4S,5R,6R)-6-(2-carboxy-5-hydroxy-3-undecylphenoxy)-3,4,5-trihydroxytetrahydro-2H-pyran-2-carboxylic acid (FGS03). The structure is made up of a long carbon chain attached to a Benzene Ring and a 2H-Pyran Ring containing a Carboxylic Acid functional group.

FIG. 3 illustrates the inhibition activity of FGS03 against Flavobacterium and recombinant human POP. FGS03 is tested at final concentrations of 0.02 mg/ml and 0.04 mg/ml respectively. Positive control is PE Inhibitor II, Calbiochem, USA. Each sample is tested in triplicate. Data represented as mean value with standard deviation, n=3.

### DETAILED DESCRIPTION OF THE INVENTION

The present invention relates to a compound, represented by formula (I):

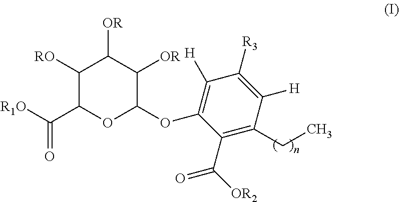

wherein
a) R, R₁, R₂ and R₄ are independently of one another a hydrogen atom or a C₁-C₆ linear or branched alkyl, or a C₁-C₆ linear or branched alkenyl, or a C₁-C₆ linear or branched alkynyl;
b) R₃ is a hydrogen atom, OR₄ or a C₁-C₆ linear or branched alkyl, or a C₁-C₆ linear or branched alkenyl, or a C₁-C₆ linear or branched alkynyl; and,
c) n=4 to 11.

The term “Cₓ-C_{y}” is to indicate C₁-C₄, C₁-C₆ or the likes, wherein x and y are integers and indicate the carbon number.

In one embodiment, the term “Cₓ-C_{y} alkyl”, when used alone or in combination with other terms, refers to a saturated hydrocarbon group that may be straight-chain or branched, having x to y carbons. In some embodiments, the alkyl group contains from 1 to 6 carbon atoms, from 1 to 5 carbon atoms, from 1 to 4 carbon atoms, from 1 to 3 carbon atoms, or 1 to 2 carbon atoms.

In one embodiment, the term “Cₓ-C_{y} alkenyl”, when used alone or in combination with other terms, refers to an alkyl group having one or more double carbon-carbon bonds that may be straight-chain or branched, having x to y carbons. In some embodiments, the alkenyl group contains 2 to 6 or 2 to 4 carbon atoms.

In one embodiment, the term “Cₓ-C_{y} alkynyl”, when used alone or in combination with other terms, refers to an alkyl group having one or more triple carbon-carbon bonds that may be straight-chain or branched, having x to y carbons. In some embodiments, the alkenyl group contains 2 to 6 or 2 to 4 carbon atoms.

Examples of alkyl moieties include, but are not limited to, chemical groups such as methyl, ethyl, n-propyl, isopropyl, n-butyl, tert-butyl, isobutyl, -butyl; higher homologs such as 2-methyl-1-butyl, n-pentyl, 3-pentyl, n-hexyl, 1,2,2-trimethylpropyl.

In another embodiment, linking substituents are described to include both the forward and backward forms of the linking substituent, wherein —O(CR′R″)ₙ— includes both —O(CR′R″)ₙ— and —(CR′R″)ₙO—. Further, if the structure contains a linking group, the Markush variable listed for the group are understood to be linking groups, wherein if the Markush variable lists “alkyl”, then it is understood that the “alkyl” represents a linking alkylene group.

In another embodiment, the compound is (2R,3S,4S,5R,6R)-6-(2-carboxy-5-hydroxy-3-undecylphenoxy)-3,4,5-trihydroxytetrahydro-2H-pyran-2-carboxylic acid (FGS03), wherein the molecular structure of the compound comprises of a long carbon chain attached to a Benzene Ring and a 2H-Pyran Ring containing a Carboxylic Acid functional group as shown in FIG. 1. The detailed information of the compound is shown in Table 1, while the properties of the compound is shown in Table 2.

TABLE 1
Detailed Information of Compound FGS03

| | |
|---|---|
| Chemical | (2R,3S,4S,5R,6R)-6-(2-carboxy-5-hydroxy-3- |
| (IUPAC) Name | undecylphenoxy)-3,4,5-trihydroxytetrahydro-2H- |
| | pyran-2-carboxylic acid |
| Chemical formula | C₂₄H₃₆O₁₀ |
| Exact mass | 484.23 |
| Molecular weight | 484.54 |
| Elemental | C 59.49; H, 7.49; O 33.02 |
| analysis | |

TABLE 2
Properties of Compound FGS03

| Properties | Values |
|---|---|
| Molar refractivity | 121.89 ± 0.3 cm³ |
| Molar Volume | 362.50 ± 0.3 cm³ |
| Parachor | 1032.9 ± 6.0 cm³ |
| Index of Refraction | 1.587 ± 0.02 |
| Surface tension | 65.9 ± 3.0 dyne/cm |
| Density | 1.336 ± 0.06 g/cm³ |
| Polarizability | 48.32 ± 0.5 × 10⁻²⁴ cm³ |
| Monoisotopic mass | 484.230847 Da |
| Nominal mass | 484 Da |
| Average mass | 484.5366 Da |
| M+ | 484.230299 Da |
| M− | 484.231396 Da |
| [M + H]+ | 485.238124 Da |
| [M + H]− | 485.239221 Da |
| [M − H]+ | 483.22274 Da |
| [M − H]+ | 483.223571 Da |

The compound FGS03 may be isolated from novel fungus strains, an example being Fusarium sp. strain F274, or may also be synthetically produced.

### Isolation and Identification of Fungus Strain

In the present embodiment of the invention, the compound was isolated from the novel fungus strain, named Fusarium sp. strain F274. The novel fungus strain was isolated from the flower of a Yam Plant, Alocasia sp. found at Kampung Semadang, Siburan, Sarawak, Malaysia. Identification of the novel fungus strain was conducted by sending the sample to the Centre for Agriculture and Bioscience International, Bakeham Lane, Egham, Surrey TW20 9TY, United Kingdom, where identification was conducted using molecular profiling via Internal Transcribed Spacer (ITS). Results of molecular profiling revealed that Fusarium solani was the closest identity to that of the novel fungus strain, but at only 94% similarity. Further identification was carried out by targeting the translation elongation factor 1-alpha (TEF) gene and the results obtained was again 94% similarity with Fusarium solani.

Fungi, which is the plural form of a fungus are a group of eukaryotic organisms that exists differently from that of the animal and plant kingdom. Commonly found in the form of a mushroom or mould, fungi can bring about many benefits to the ecosystem by decomposing dead matter and creating a recyclable source of nutrients. Moreover, fungi are known to survive harsh conditions, such as bad weather and tough terrain, allowing the cultivation of these fungi to be an easy process. While some fungi are also known to be pathogenic in nature, there are also some that are known to have medicinal properties due to metabolites present within the fungi. Hence, the cultivation of fungi for the purpose of producing beneficial medication is proven to be promising, as it brings about more advantages when compared to plants, such as fast mature rate and higher yield.

### Usage of Compound

As mentioned above, POP is a ubiquitous post-proline cleaving enzyme that is highly expressed in the brain and facilities several functions of the central nervous system such as memory, mood and learning. POP functions by cleaving short peptides (<30 amino acids) with a high specific cleavage at the carboxyl side of proline.

In an embodiment of the invention, the compound is able to inhibit POP enzyme, and by inhibiting POP enzyme, the compound may be used to treat neurodegenerative diseases such as Parkinson's disease of Alzheimer's disease. POP functions by cleaving short peptides (<30 amino acids) with a high specific cleavage at the carboxyl side of proline. Furthermore, POP can also modulate the functions of protein partners such as neuronal peptides and hormones containing a proline residue. As many biologically active compounds contain proline, mainly neuropeptides, by inhibiting the activity of POP, the symptoms of these neurodegenerative diseases can be suppressed. To-date, the active sites of POP enzyme can be further grouped into several subsites, as shown in the table below.

TABLE 3
Subsites of Active Sites of POP Enzyme

| Subsite | Formed by Side Chain | Function |
|---|---|---|
| S1 Specificity | Phe476, Asn555, Val580, | Form a hydrophobic |
| Pocket | Trp595, Tyr599, and | environment for the easy fit |
| | Val644 | of the substrate proline or |
| | | the aromatic rings of the |
| | | inhibitors |
| S2 Specificity | Arg643 | Comparably less specific |
| Pocket | | |
| S3 Specificity | Phe173, Met235, Cys255, | Creates a relatively large |
| Pocket | Ile591, and Ala594 | hydrophobic environment |

Furthermore, studies have also shown that POP inhibitors target the active site Tyr599 (Subsite S1 specificity pocket), ARG 643 (Subsite S2 specificity pocket), Phe173 (Subsite S3 specificity pocket), and Ser554 on the POP enzyme.

### Isolation and Identification of Compound

The present invention further relates to a process for isolating a novel compound (2R,3S,4S,5R,6R)-6-(2-carboxy-5-hydroxy-3-undecylphenoxy)-3,4,5-trihydroxytetrahydro-2H-pyran-2-carboxylic acid (FGS03) through a series of steps comprising fermenting, extracting, fractioning, and purifying the novel fungus strain isolated from the flower.

### Fermenting Step

A fungus, Fusarium sp. strain F274 isolated from the flower of a Yam Plant, Alocasia sp. found at Kampung Semadang, Siburan, Sarawak, Malaysia is fermented using a fermentation medium. The fermentation medium comprises 10 g of glucose, 0.55 g monosodium glutamate, 0.5 g yeast extract, 0.13 g monopotassium phosphate, 0.2 g magnesium sulfate, 0.2 g sodium sulphate decahydrate, 0.07 g dipotassium phosphate, 0.02 g iron(II) sulfate heptahydrate, 0.01 g manganese(II) sulfate, 0.002 g zinc sulphate heptahydrate and 0.002 g copper sulphate heptahydrate. 10% (v/v) of the fungus is inoculated in flasks containing the fermentation medium and incubated at a temperature of 35° C. to 37° C. via agitation or agitated using a rotary shaker at 180 rpm for up to 7 days.

### Extracting Step

The fermentation medium used for fermenting the fungus was then added with an equal volume of polar organic solvents selected from one or a combination of n-butyl alcohol, isopropyl alcohol, n-butyl acetate, isobutyl alcohol, methyl isoamyl ketone, n-propyl alcohol, tetrahydrofuran, chloroform, methyl isobutyl ketone, ethyl acetate, methyl n-propyl ketone, methyl ethyl ketone or 1,4-Dioxane to form a mixture. The mixture was then mechanically agitated by using a shaker for up to 1 hour before separating the organic solvent layer using a centrifuge machine spun at up to 4000 rpm. The organic solvent layer was then dried by using a concentrator (SpeedVac™) to produce a dried crude extract, whereby the dried crude extract is resuspended using 1 mL of 10% organic solvent such as dimethyl sulfoxide (DMSO) for further test.

### Fractioning Step

This process involves fractioning the dried crude extract followed by purification, wherein liquid chromatography system selected from one or a combination of liquid-solid chromatography, normal phase chromatography, high-performance liquid chromatography, reverse phase chromatography, flash chromatography, partition chromatography, ion chromatography, size exclusion chromatography, supercritical fluid chromatography, affinity chromatography or chiral chromatography was used as the stationary phase, while the mobile phase was chosen from one or a combination of hexane, dichloromethane, ethyl acetate or methanol.

The detailed fractioning process is as such, where 8 g of dried crude extract which was obtained from 80 litres of organic solvent from the previous fermenting step is subjected to normal phase column chromatography (ID 5 cm, 50 cm height) with a stationary phase volume of 800 cm³, and a gradient mobile phase of increasing polarity from hexane, dichloromethane, ethyl acetate to methanol. The crude extract was mixed with 12 g of celite powder and dry loaded into the open column. Fractions were collected at 150 cm³ interval while the solvent system is changed according to visual observation on the elution of compounds in the crude extract. The mobile phase mentioned is based on a solvent system described in the table below.

TABLE 4
Solvent System used in the Fractioning Process

| Solvent | Ratio | Volume |
|---|---|---|
| Hexane | 100% | 1.5 L |
| Hexane:Dichloromethane | 7:3 | 1.5 L |
| | 1:1 | 1.0 L |
| | 3:7 | 1.5 L |
| Dichlromomethane | 100% | 1.0 L |
| Dichloromethane:Ethyl Acetate | 8:2 | 1.0 L |
| | 6:4 | 1.0 L |
| | 1:1 | 1.0 L |
| | 4:6 | 1.0 L |
| | 2:8 | 1.0 L |
| Ethyl Acetate | 100% | 1.0 L |
| Ethyl Acetate:Methanol | 8:2 | 0.8 L |
| | 6:4 | 0.6 L |
| | 4:6 | 0.7 L |
| | 2:8 | 1.1 L |
| Methanol | 100% | 3.0 L |

A total of 98 fractions were collected and pooled to 28 fractions after chemical profiling was done using High Performance Liquid Chromatography analysis. The pooled 28 fractions were then subjected to bioassay analysis to determine the potency of the fraction in inhibiting POP enzyme activity.

### Purifying Step

The purifying step is done by collecting fractions from the fractioning step that showed the most potent bioassay results and subjecting them to purification using a High Performance Liquid Chromatography (Agilent 1200 series) with a gradient solvent system, whereby the detailed method used for purification is shown in the table below. This process is repeated until the purity is approximately 95% or more. In the present embodiment, this process was repeated 2-3 times. Subsequently, recrystallisation by redissolving the compound in methanol with subsequent slow evaporation yielded pure FGS03.

TABLE 5
High Performance Liquid Chromatography Method for Purification

| | |
|---|---|
| Method: | 10-100_MEOH_SLOW_GRADIENT_F274_ |
| | PURIF.M |
| Solvent System: | 90% water, 10% methanol to 100% methanol |
| | 0 minute = 10% methanol |
| | 5^{th} minute = 70% methanol |
| | 10^{th} Minute = 95% methanol |
| | 40^{th} minute = 100% methanol |
| | * 5 minutes post run time |
| Injection Volume: | 80 μL |
| Flow Rate: | 2.5 mL/min |
| Column used: | Agilent Eclipse XDB-C18, 5 um, 9.4 × 250 mm |

The purified compound was identified using Nuclear Magnetic Resonance (NMR), whereby the results are shown below. The results from the NMR indicated that the structure is made up of a long carbon chain attached to a Benzene Ring and a 2H-Pyran Ring containing a Carboxylic Acid as observed in FIG. 1.

TABLE 6
Nuclear Magnetic Resonance Correlation Table of FGS03.

| | FGS03 | |
|---|---|---|
| Position | δ_{H}, mult. (J in Hz) | δ_{C} |
| 1 | | 174.69 |
| 2 | | 108.73 |
| 3 | | 149.81 |
| 4 | 6.45, s | 112.25 |
| 5 | | 165.84 |
| 6 | 6.43 | 102.81 |
| 7 | | 162.53 |
| 8 | 2.91, m/t | 37.43 |
| 9 | 1.56, m | 33.21 |
| 10 | 1.29, s | 33.18 |
| 11 | 2.16, s; 1.29, s | 30.98 |
| 12 | 1.29, s | 30.95 |
| 13 | 1.29, s; 1.32, s | 30.88 |
| 14 | 1.29, s; 1.32, s | 30.85 |
| 15 | 1.29, s; 1.32, s | 30.69 |
| 16 | 1.29, s; 1.32, s | 30.57 |
| 17 | 1.32, s | 23.84 |
| 18 | 0.89, m/t | 14.58 |
| 1′ | | 172.68 |
| 2′ | 4.00 (9.6) | 76.67 |
| 3′ | 3.61, m | 73.10 |
| 4′ | 3.47, m | 74.55 |
| 5′ | 3.50, m | 77.37 |
| 6′ | 5.03, d/m | 101.39 |

*¹H NMR spectra and ¹³C NMR spectra were measured at 400 MHz

### Testing of Compound

The POP inhibitory activity of compound was evaluated using an enzyme inhibition assay. A colorimetric assay was set up using Flavobacterium and recombinant human POP enzymes. The POP enzyme solutions obtained from Flavobacterium (0.5 U mL⁻¹ giving 0.0416 units per well) and human (USBiological, MA with working concentration 0.006 mg/ml) were added to the 96-well plates containing sample extracts and phosphate buffer (100 mM, pH 7.0). The reaction was initiated with the addition of enzyme substrate, Z-Gly-Pro-4-nitroanilide (2 mM in 40% dioxane) and incubated at 30° C. for 15 minutes. Stopper buffer (2 M acetate buffer, pH 4.0 with 10% Triton-X) was added to stop the enzyme reaction. The released p-nitroanilide was determined colorimetrically with a plate reader at 414 nm. The inhibition activity was calculated using the formula:

$$\\ Percentage of inhibition = 1 - ( \frac{OD sample}{OD blank} ) \times 100 \% ,$$

where blank=control without the inhibitor
OD=change in optical density during the assay.

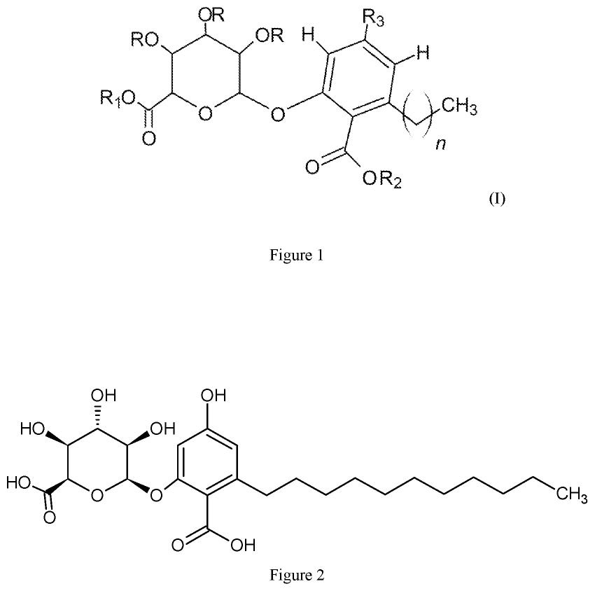

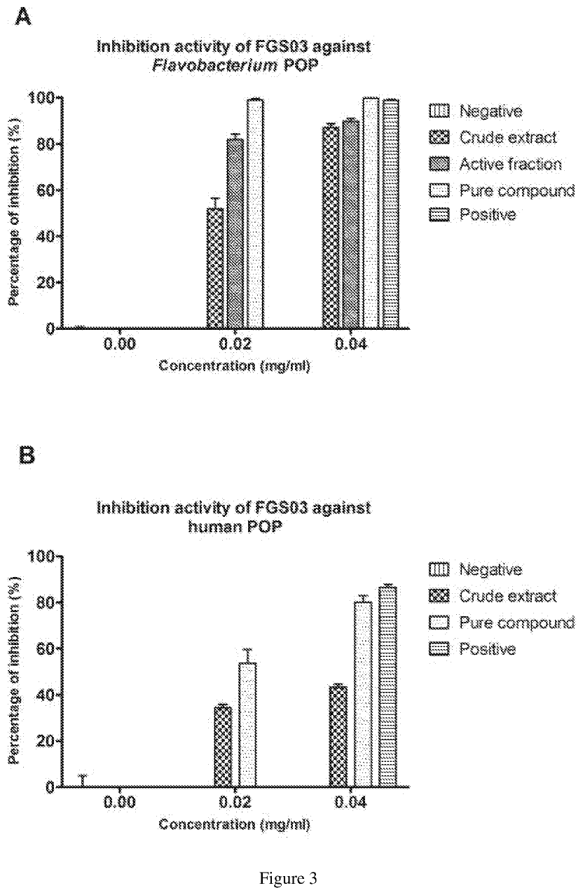

The POP inhibitory assay was performed for crude extract, active fraction and purified compound of FGS03. From the results shown in FIG. 2, it can be clearly seen that when compared to the positive control, there was already a significant percentage of inhibition by the crude extract (87.06±1.64%) indicating that the presence of a compound that has inhibitory activity from the fungus, Fusarium sp. strain F274. Moreover, the further process of fractioning and purifying resulted in a pure compound that shows similar inhibitory activity as compared to that of the positive control, indicating that the compound FGS03 can be used as an inhibitor for POP enzyme. Similarly, when tested against POP enzyme from human, the compound shows potent inhibition. The inhibition is seen at 80% at 0.04 mg/ml against human POP.

Hence, the results indicate that the compound FGS03 isolated from the fungus Fusarium sp. strain F274 was able to inhibit POP significantly and may be a viable option for treating, or suppressing the symptoms of neurodegenerative diseases in people suffering from said diseases. Moreover, compound FGS03 may also be formulated and manufactured into a medicament or formulation for the treatment of neurodegenerative diseases.

Further mechanistic studies were also done using molecular docking of FGS03 towards human POP enzyme (3DDU) via the Chimera Autodock with Vina software. Results indicated that FGS03 had 4 hydrogen bond interaction with the POP enzyme at the active sites, His680, Arg643 (Subsite S2 specificity pocket), Act801 and Phe476. Moreover, the presence of aromatic phenoxycarboxylic acid would also enable FGS03 to fit into the Si specificity pocket of the active site that provides a hydrophobic environment for the easy fit of the substrate proline or the aromatic rings of the inhibitors. In addition, the carbon chain of FGS03 would be able to fit into the S3 specificity pocket of 3DDU active site, as the specificity pocket creates a relatively large hydrophobic environment.

Although the present invention has been described in a specific embodiment as in the above description, it is understood that the above description does not limit the invention to the above given details. It will be apparent to those skilled in the art that various changes and modification may be made therein without departing from the principle of the invention or from the scope of the appended claims.

### REFERENCES

1. Männistö P T, García-Horsman J A. Mechanism of Action of Prolyl Oligopeptidase (PREP) in Degenerative Brain Diseases: Has Peptidase Activity Only a Modulatory Role on the Interactions of PREP with Proteins?. Front Aging Neurosci. 2017; 9:27. doi:10.3389/fnagi.2017.00027
2. Männisto P T, Venäläinen J, Jalkanen A, García-Horsman JA. Prolyl oligopeptidase: a potential target for the treatment of cognitive disorders. Drug News Perspect. 2007; 20(5):293-305. doi:10.1358/dnp.2007.20.5.1120216.
3. Park Y S, Jang H J, Lee K H, Hahn T R, Paik Y S. Prolyl endopeptidase inhibitory activity of unsaturated fatty acids. J Agric Food Chem. 2006; 54(4):1238-1242. doi:10.1021/jf052521h.

1. A compound, represented by formula (I): 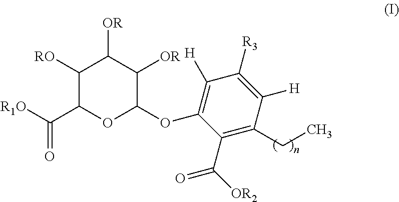
wherein
a) R, R₁, R₂ and R₄ are independently of one another a hydrogen atom or a C₁-C₆ linear or branched alkyl, or a C₁-C₆ linear or branched alkenyl, or a C₁-C₆ linear or branched alkynyl;
b) R₃ is a hydrogen atom, OR₄ or a C₁-C₆ linear or branched alkyl, or a C₁-C₆ linear or branched alkenyl, or a C₁-C₆ linear or branched alkynyl; and,
c) n=4 to 11.

2. The compound according to claim 1, wherein the compound is (2R,3S,4S,5R,6R)-6-(2-carboxy-5-hydroxy-3-undecylphenoxy)-3,4,5-trihydroxytetrahydro-2H-pyran-2-carboxylic acid (FGS03).

3. The compound according to claim 2, wherein the compound is isolated from a fungus, Fusarium sp. strain F274.

4. The compound according to claim 3, wherein the fungus is isolated from a flower of a Yam plant, Alocasia sp.

5. The compound according to claim 2, wherein the compound is produced by chemical synthesis.

6. The compound according to claim 2, wherein the compound inhibits Prolyl Oligopeptidase enzyme.

7. The compound according to claim 2 wherein the compound is used for treating neurodegenerative diseases by inhibiting Prolyl Oligopeptidase enzyme.

8. Use of compound according to claim 2 for the preparation of a medicament for the treatment of neurodegenerative diseases.

9. The compound according to claim 7, wherein the neurodegenerative diseases comprise Parkinson's disease or Alzheimer's disease.

10. A process for isolating a compound (2R,3S,4S,5R,6R)-6-(2-carboxy-5-hydroxy-3-undecylphenoxy)-3,4,5-trihydroxytetrahydro-2H-pyran-2-carboxylic acid (FGS03), comprising the steps of:
a) Fermenting a fungus, Fusarium sp. strain F274 using a fermentation medium;
b) Extracting the compound from the fermentation medium of step (a) using a first organic solvent;
c) Fractioning the compound of step (b) using a first stationary phase and a first mobile phase; and,
d) Purifying the compound of step (c) using a second stationary phase and a second mobile phase.

11. The process according to claim 10, comprising fermenting 10% (v/v) of the fungus in the fermentation medium at 35° C. to 37° C. for 7 days through agitation.

12. The process according to claim 10, wherein the fermentation medium comprises:
a) glucose;
b) monosodium glutamate;
c) yeast extract;
d) monopotassium phosphate;
e) magnesium sulfate;
f) sodium sulphate decahydrate;
g) dipotassium phosphate;
h) iron(II) sulfate heptahydrate;
i) manganese(II) sulfate;
j) zinc sulphate heptahydrate; and,
k) copper sulphate heptahydrate.

13. The process according to claim 10, comprising equal volumes of the first organic solvent mixed with equal volume of the fermentation medium to form a mixture.

14. The process according to claim 10, wherein the first organic solvent comprises one or a combination of n-butyl alcohol, isopropyl alcohol, n-butyl acetate, isobutyl alcohol, methyl isoamyl ketone, n-propyl alcohol, tetrahydrofuran, chloroform, methyl isobutyl ketone, ethyl acetate, methyl n-propyl ketone, methyl ethyl ketone or 1,4-Dioxane.

15. The process according to claim 13, further comprising agitating the mixture for 1 hour and separating the first organic solvent from the mixture.

16. The process according to claim 10, wherein the first and the second stationary phase comprises a liquid chromatography system.

17. The process according to claim 16, wherein the liquid chromatography system comprises one or a combination of liquid-solid chromatography, normal phase chromatography, high-performance liquid chromatography, reverse phase chromatography, flash chromatography, partition chromatography, ion chromatography, size exclusion chromatography, supercritical fluid chromatography, affinity chromatography or chiral chromatography.

18. The process according to claim 10, wherein the first mobile phase and second mobile phase comprises a second organic solvent.

19. The process according to claim 18, wherein the second organic solvent comprises one or a combination of hexane, dichloromethane, ethyl acetate or methanol.

20. The process according to claim 10, wherein the purifying process is repeated until purity of the compound is more or equal to 95%.
